(19) United States
(12) Patent Application Publication
(10) Pub. No.: US 2025/0391531 A1
(43) Pub. Date: Dec. 25, 2025

(54) MEDICAL REPORTER, MEDICAL REPORTING SYSTEM, AND MEDICAL REPORTING METHOD

(71) Applicants: CANON MEDICAL SYSTEMS CORPORATION, Otawara-shi (JP); INSTITUTE OF SCIENCE TOKYO, Tokyo (JP)

(72) Inventors: Kazumasa NORO, Shioya-gun (JP); Yuka MATSUMURA, Shioya-gun (JP); Ryo UCHIMIDO, Meguro (JP); Kenji WAKABAYASHI, Meguro (JP)

(73) Assignees: CANON MEDICAL SYSTEMS CORPORATION, Otawara-shi (JP); INSTITUTE OF SCIENCE TOKYO, Tokyo (JP)

(21) Appl. No.: 19/244,101

(22) Filed: Jun. 20, 2025

(30) Foreign Application Priority Data
Jun. 20, 2024 (JP) 2024-099930

(51) Int. Cl. G16H 15/00; G16H 10/60

(52) U.S. Cl. CPC G16H 15/00; G16H 10/60

(57) ABSTRACT

A medical reporter according to an embodiment includes circuitry. The circuitry extracts a word element, from recorded data in which the content of statements made by a plurality of speakers on a subject is recorded; based on the concept indicated by each of a plurality of the extracted word elements, specifies a word attribute for each of the word elements; based on at least one of the word attribute, a speaker attribute of a speaker according to the word element, a temporal relation between time related to the content of the statement including the word element and reference time, and medical resource information on a medical resource according to the word element, calculates an importance degree of each of the word elements; and based on the importance degree of each of the word elements, presents a candidate for a medical procedure to be performed on the subject.

### CROSS-REFERENCE TO RELATED APPLICATIONS

This application is based upon and claims the benefit of priority from Japanese Patent Application No. 2024-099930, filed on Jun. 20, 2024, the entire contents of which are incorporated herein by reference.

### FIELD

Embodiments described herein relate generally to a medical reporter, a medical reporting system, and a medical reporting method.

### BACKGROUND

Conventionally, in intensive care units (ICUs) of medical facilities such as hospitals, one patient may be cared for 24-hours a day by multiple professional staff. In such a care, timely collaboration and information sharing among multiple professionals are important. Thus, in general, in the ICU, multiple professional staff get together to conduct multidisciplinary rounds to determine the patient care policies on the day (set a ToDo list).

### BRIEF DESCRIPTION OF THE DRAWINGS

FIG. 1 is a diagram illustrating an example of a configuration of a medical information processing system according to an embodiment;

FIG. 2 is a diagram for explaining an example of a generation process of text data according to the embodiment;

FIG. 3 is a diagram for explaining an example of word segmentation according to the embodiment;

FIG. 4 is a diagram for explaining an example of a fluctuation correction process according to the embodiment;

FIG. 5 is a diagram for explaining an example of an attribute specification process according to the embodiment;

FIG. 6 is a diagram for explaining an example of a calculation process of an importance degree according to the embodiment;

FIG. 7 is a diagram illustrating an example of a presentation screen of a ToDo list according to the embodiment; and

FIG. 8 is a flowchart illustrating an example of a process executed by a medical information processing apparatus according to the embodiment.

### DETAILED DESCRIPTION

A medical reporter according to an embodiment includes circuitry.

The circuitry receive recorded data made by a plurality of speakers on a subject is recorded, extract a word element, from recorded data in which content of a statement, output extracted recorded data by using the recorded data which includes extracted word element with specified speaker, specify, by using the extracted recorded data, and based on a concept indicated by each of a plurality of the extracted word elements, a word attribute for each of the word elements. And the circuitry calculates, based on at least one of the word attribute, a speaker attribute of a speaker according to the word element, a temporal relation between time related to the content of the statement including the word element and reference time, and medical resource information on a medical resource according to the word element, an importance degree of each of the word elements, and generates a report data based on the importance degree of each of the word elements, which presenting a candidate for a medical procedure to be performed on the subject.

Hereinafter, embodiments of a medical reporter, a medical reporter system, and a medical reporting method will be described in detail with reference to the accompanying drawings. In the following embodiments, the medical reporter may be described or addressed as a medical information processing system.

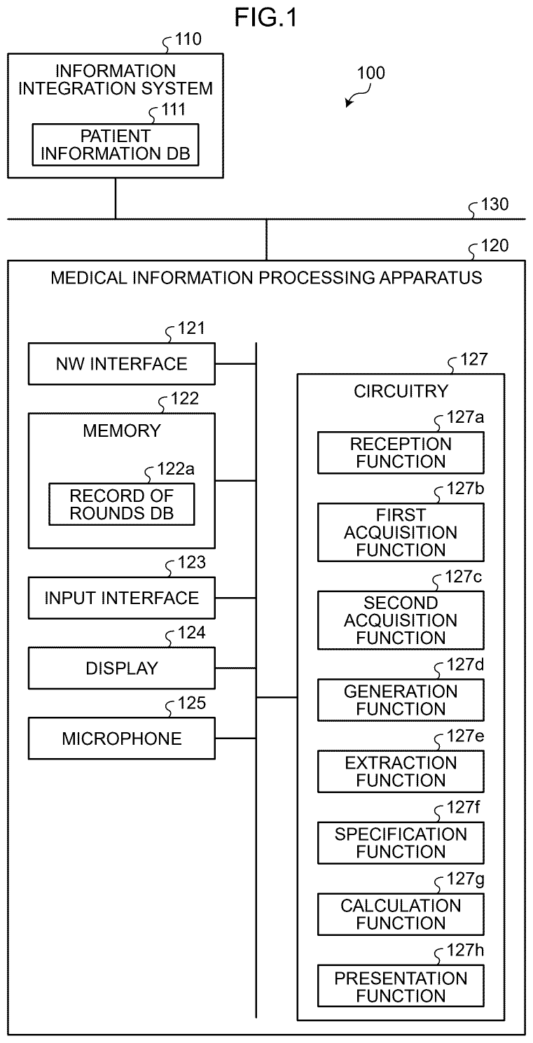

FIG. 1 is a diagram illustrating an example of a configuration of a medical information processing system according to the present embodiment.

The medical information processing system according to the present embodiment is installed in medical facilities such as hospitals, and effectively records and shares information important for determining the next medical procedure to be performed on a subject.

Specifically, in a meeting held as part of multidisciplinary rounds (hereinafter, may simply be referred to as a meeting), the medical information processing system according to the present embodiment extracts a word element from the statements of the participants, calculates the importance degree of the word element, and presents the medical procedure derived from the word element with a high importance degree, to a user as a candidate (ToDo list) for the next medical procedure to be performed on the subject.

For example, as illustrated in FIG. 1, a medical information processing system 100 according to the present embodiment includes an information integration system 110 and a medical information processing apparatus 120. In this example, the information integration system 110 and the medical information processing apparatus 120 are communicatively connected to each other via a network 130 such as a local area network (LAN) or a wide area network (WAN).

The information integration system 110 includes a patient information database (DB) 111. For example, the information integration system 110 is implemented by a computer device such as a server and a workstation including an electronic chart system, a hospital information system (HIS), a radiology information system (RIS), and the like.

The patient information DB 111 stores various types of medical information on a patient. A patient is an example of a subject. For example, of a plurality of patients, the patient information DB 111 stores information such as disease (pre-existing disease, complication, side effect, and the like) and symptoms (conditions) with respect to the treatment (including administered drugs) of each of the patients, and connected devices as medical information.

The medical information processing apparatus 120 is an apparatus used by a health care worker such as a doctor who determines the medical procedure to be performed on a patient. The medical information processing apparatus 120 has a function of presenting a medical procedure to be performed on the patient, on the basis of the statements made by the participants of the meeting. For example, the medical information processing apparatus 120 is implemented by a personal computer and the like.

Specifically, the medical information processing apparatus 120 includes a network (NW) interface 121, a memory 122, an input interface 123, a display 124, a microphone 125, and circuitry 127.

The NW interface 121 controls transmission and communication of various types of data transmitted and received between another apparatus connected via the network 130 and the medical information processing apparatus 120. Specifically, the NW interface 121 is connected to the circuitry 127, and outputs information received from the information integration system 110 to the circuitry 127. For example, the NW interface 121 is implemented by a network card, a network adapter, a network interface controller (NIC), and the like.

The memory 122 stores various types of data, various computer programs, and the like. For example, the memory 122 is implemented using a semiconductor memory element such as a random access memory (RAM) and a flash memory, a hard disk, an optical disc, or the like.

Specifically, the memory 122 includes a record of rounds DB 122a. The record of rounds DB 122a stores information on the record of rounds in which the statements of participants of the meeting and a ToDo list are recorded.

The input interface 123 receives input operations of various instructions and various types of information from an operator. Specifically, the input interface 123 is connected to the circuitry 127, and converts the input operation received from the operator into an electrical signal and transmits the converted electrical signal to the circuitry 127.

For example, the input interface 123 is implemented using a trackball, a switch button, a mouse, a keyboard, a touch pad with which input operations are performed by touching an operation surface, a touch screen in which a display screen and a touch pad are integrated, a non-contact input circuit using an optical sensor, a voice input circuit, and the like.

In the present specification, the input interface 123 is not limited to one having physical operation parts such as a mouse and a keyboard. For example, circuitry of electrical signals that receives an electrical signal corresponding to an input operation from an external input device, which is provided separately from the apparatus, and that outputs the electrical signal to the control circuit, is also included in an example of the input interface 123.

The display 124 displays various types of information. Specifically, the display 124 is connected to the circuitry 127, and displays various types of information output from the circuitry 127. For example, the display 124 is implemented using a liquid crystal monitor, a cathode ray tube (CRT) monitor, a touch panel, and the like.

The microphone 125 records the voice of the participants during the meeting. A plurality of the microphones 125 may be provided. For example, the microphone 125 may be provided for each of the participants in the meeting.

The circuitry 127 controls components of the medical information processing apparatus 120. Specifically, the circuitry 127 includes a reception function 127a, a first acquisition function 127b, a second acquisition function 127c, a generation function 127d, an extraction function 127e, a specification function 127f, a calculation function 127g, and a presentation function 127h.

The reception function 127a receives input of various operations from a user. For example, the reception function 127a receives input to designate a target patient to whom multidisciplinary rounds are to be performed from the user, via the input interface 123. Specifically, the reception function 127a receives input of the patient ID of the target patient from the user. The reception function 127a may also receive input to select the patient ID of the target patient, from a plurality of patient IDs displayed in a list format from the user.

Moreover, for example, the reception function 127a receives input to start the meeting from the user. Specifically, the reception function 127a receives input to press the meeting start button displayed on the display 124 from the user.

Moreover, for example, the reception function 127a receives input to terminate the meeting from the user. Specifically, the reception function 127a receives input to press the meeting termination button displayed on the display 124 from the user.

The first acquisition function 127b acquires patient information on the target patient. For example, the first acquisition function 127b refers to the patient information DB 111 of the information integration system 110 via the NW interface 121 and the network 130, and acquires the patient information corresponding to the patient ID received by the reception function 127a. Specifically, the first acquisition function 127b acquires information such as medical department, name, age, the number of days admitted to the ICU, disease, purpose of admission, and treatment of the target patient. In the following embodiment, admission to the ICU is used as an example. However, the number of days admitted to the hospital, and the date when a specific treatment has started may also be input.

The second acquisition function 127c acquires voice data in which the content of statements made by a plurality of participants of the meeting on the patient is recorded. The voice data is an example of recorded data. For example, the second acquisition function 127c acquires voice data recorded by the microphone 125, when the reception function 127a receives input to start the meeting. Moreover, when the reception function 127a receives input to terminate the meeting, the second acquisition function 127c terminates the process of acquiring voice data. The statements of participants in the voice data are distinguished and recorded for each of the speakers. If the microphone 125 is provided for each of the participants, the participants can be distinguished by determining which microphone 125 was used to collect the statement. Moreover, by using voice recognition and the like, the voice of the participant can be distinguished from video data in which the voice of the participants are mixed.

If the meeting is conducted in a chat format using terminal devices such as smartphones and tablet terminals, the second acquisition function 127c may acquire text data input by the participants of the meeting via the terminal devices, as recorded data.

The generation function 127d generates text data, on the basis of voice data. For example, the generation function 127d generates text data, by converting the voice data acquired by the second acquisition function 127c into text data, using known voice recognition techniques.

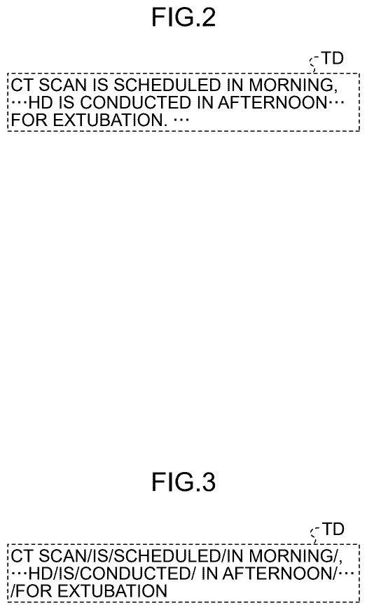

Herein, FIG. 2 is a diagram for explaining an example of a generation process of text data. As illustrated in FIG. 2, text data TD in which the statement of the participant of the meeting is represented by text is generated. In the example in FIG. 2, the text data TD is generated by converting the statement of the participant of the meeting of “CT scan is scheduled in the morning, . . . . HD is conducted in the afternoon . . . for extubation” into text.

Returning to FIG. 1, the description will continue. The extraction function 127e extracts word elements from the generated text data. For example, the extraction function 127e segments a sentence included in the text data TD generated by the generation function 127d into words, using known natural language processing techniques.

Herein, FIG. 3 is a diagram for explaining an example of word segmentation. As illustrated in FIG. 3, the sentence included in the text data TD is segmented into words. In the example in FIG. 3, the sentence of “CT scan is scheduled in the morning, . . . . HD is conducted in the afternoon . . . for extubation” is segmented into words by dividing the sentence into “CT scan”, “is”, “scheduled”, “in the”, “morning”, . . . , “HD”, “is conducted”, “in the”, “afternoon”, . . . , “for extubation”.

Moreover, for example, for the segmented words, the extraction function 127e performs fluctuation correction, and integrates synonyms into a single expression.

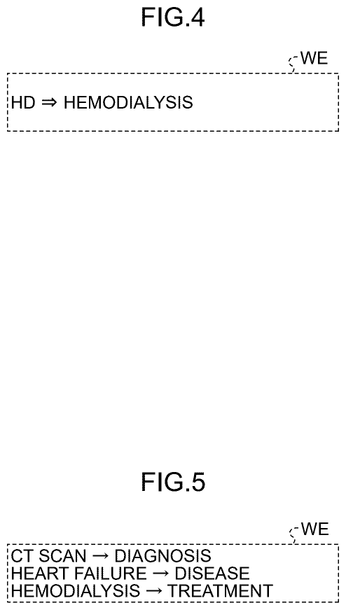

Herein, FIG. 4 is a diagram for explaining an example of fluctuation correction. As illustrated in FIG. 4, words having the same meaning such as “HD” and “hemodialysis” are integrated into a single expression. As an example, the extraction function 127e integrates synonyms into the expression used most frequently. In FIG. 4, “HD” is converted into “hemodialysis”. In the example in FIG. 4, “hemodialysis” is a word element WE the importance degree of which is to be calculated, which will be described below. The expression integration can be performed, by holding a dictionary file in which synonyms are arranged in advance, and determining whether the segmented word corresponds to the synonym.

The extraction function 127e extracts a plurality of the word elements WE from the text data generated by the generation function 127d, by performing the word segmentation and fluctuation correction described above.

Returning to FIG. 1, the description will continue. The specification function 127f specifies the word attribute of each of the word elements, on the basis of the concept indicated by each of the extracted word elements. For example, by using known medical ontologies and the like, the specification function 127f specifies the word attribute for the word element, by conceptualizing each of the extracted word elements to a higher level.

As an example, if the word element “hemodialysis” is extracted, by using a known medical ontology, the specification function 127f acquires hypernyms in the medical ontology, such as “hemodialysis”→“dialysis therapy”→“treatment”. Moreover, similarly, if the word element “enteral nutrition” is extracted, the specification function 127f acquires hypernyms such as “enteral nutrition”→“nutrition control”→“treatment”. Furthermore, similarly, if the word element “CT” is extracted, the specification function 127f acquires a hypernym such as “CT”→“diagnosis”.

Still furthermore, similarly, if the word element “respiratory rate” is extracted, the specification function 127f acquires hypernyms such as “respiratory rate”→“vital sign”→“physical examination”. Still furthermore, similarly, if the word element “heart failure” is extracted, the specification function 127f acquires a hypernym such as “heart failure”→“disease”. Still furthermore, similarly, if the word element “edema” is extracted, the specification function 127f acquires a hypernym such as “edema”→“symptoms”.

Herein, FIG. 5 is a diagram for explaining an example of an attribute specification process. As illustrated in FIG. 5, for the word element WE of “CT scan”, by using a known medical ontology, the specification function 127f acquires “diagnosis”, that is a hypernym in the medical ontology. Similarly, for the word element WE of “heart failure”, the specification function 127f acquires “disease” that is a hypernym. Moreover, similarly, for the word element WE of “hemodialysis”, the specification function 127f acquires “treatment” that is a hypernym.

Then, according to the conversion results, the specification function 127f specifies that the word attribute of the “CT scan” is “diagnosis”, the word attribute of the “heart failure” is “disease”, and the word attribute of the “hemodialysis” is “treatment”.

Returning to FIG. 1, the description will continue. The calculation function 127g calculates the importance degree of the word element. For example, the calculation function 127g calculates the importance degree, using the appearance frequency of the word element, the number of times the medical procedure according to the word element was used in the ToDo list in the past, or the like, as an index.

Specifically, the calculation function 127g calculates the importance degree so that the value of the importance degree increases, as the appearance frequency of the word element during the meeting increases. Moreover, the calculation function 127g analyzes the past data using a statistical method, and calculates the importance degree so that the value of importance degree increases, as the number of times the medical procedure according to the word element was used in the ToDo list in the past increases.

Furthermore, for example, the calculation function 127g corrects the importance degree of the calculated word element, while taking into account the word attribute of the word element, the speaker attribute of the speaker according to the word element, the temporal relation between the time related to the content of a statement including the word element and the reference time, the medical resource information of medical resources according to the word element, the seriousness of the disease when the derived word element is disease, and the like. In the following embodiments, the correction is made by first calculating the importance degree from the appearance frequency of the word element, for example. However, it is not limited thereto. Instead of using the appearance frequency, the importance degree may also be calculated directly, using the word attribute of the word element, the speaker attribute, the time relation, the medical resource information, and the seriousness.

In the present embodiment, the calculation function 127g sets first weight to be used for calculating the importance degree, according to the word attribute of the word element. Specifically, the calculation function 127g analyzes the past data using a statistical method, and sets the first weight so that the value of the first weight increases, as the number of times the medical procedure according to the word element of the word attribute was used in the ToDo list in the past increases.

Herein, the speaker attribute is information indicating the attribute of the speaker who made a statement on the extracted word element. As an example, the speaker attribute is information indicating the work category of the speaker such as a doctor in charge, an ICU doctor, an ICU nurse, a pharmacist, a nutritionist, a physical therapist, and a clinical engineer.

Known voice recognition techniques and the like may be used for specifying the speaker. For example, by using a statistical method, a machine learning method, and the like, the calculation function 127g extracts features of voice of the speaker such as the pitch of voice, sounding of the voice, speaking speed, and the speech habit, from the voice data acquired by the second acquisition function 127c, and specifies the speaker on the basis of the extracted features.

Moreover, the calculation function 127g may specify the speaker, by providing a plurality of the microphones 125, associating identification information on the speaker with each of the microphones 125 prior to the meeting, and specifying which microphone 125 was used to record the voice data.

In the present embodiment, the calculation function 127g sets second weight to be used for calculating the importance degree, according to the speaker attribute. Specifically, the calculation function 127g analyzes the past data using a statistical method, and sets the second weight so that the value of the second weight increases, as the number of times the medical procedure according to the word element stated by the speaker of the speaker attribute was used in the ToDo list in the past increases.

In this manner, by calculating the importance degree while taking into account the speaker attribute, for example, even if a speaker, whose work category rarely takes part in setting a ToDo list, often states the word relating to the medical procedure during the meeting, the medical procedure based on the word is less likely presented as a ToDo list. Therefore, with the medical information processing apparatus 120 according to the present embodiment, it is possible to present more appropriate medical procedure as a ToDo list.

Moreover, the information is information indicating the temporal relation between the time related to the extracted word element and the reference time. For example, the reference time is the time point when the importance degree is calculated.

As an example, if text data including a sentence of “Yesterday, hemodialysis was performed” is generated, “Yesterday” functions as the time information on the word element “hemodialysis”. Similarly, in the text data of “scheduled to perform CT scan this afternoon”, “this afternoon” is the time information of the word element “CT scan”. Moreover, similarly, in the text data of “extubation after the condition becomes stable”, “after the condition becomes stable” is the time information of the word element “extubation”.

In the present embodiment, according to the time information, the calculation function 127g sets third weight to be used for calculating the importance degree. Specifically, if the time information represents before the time point when the importance degree is calculated, the calculation function 127g sets the value of the third weight to be small.

Moreover, if the time information represents after the time point when the importance degree is calculated, the calculation function 127g sets the third weight so that the value of the third weight increases, as the time indicated by the time information is closer to the time point when the importance degree is calculated.

Furthermore, if the time information includes a condition, the calculation function 127g sets the third weight according to the achievement status of the condition.

Specifically, if the time information includes the condition of “after the condition becomes stable”, the calculation function 127g specifies the condition of the patient, on the basis of the text data generated by the generation function 127d. If the word element of the context indicating that the condition of the patient is unstable appears in the text data at a frequency equal to or greater than a predetermined threshold value, the calculation function 127g sets the third weight so that the value of the third weight becomes small. On the other hand, on contrary, if the word element of the context indicating that the condition of the patient is stable appears in the text data at a frequency equal to or greater than a predetermined threshold value, and when it is possible to assume that the condition of the patient seems to become stable in near future, or when the condition has become stable, the calculation function 127g sets the third weight so that the value of the third weight increases.

In this manner, by calculating the importance degree while taking into account the time information or future prediction, for example, even if the word relating to the medical procedure that has already been performed appears frequently, the medical procedure based on the word is less likely presented as a ToDo list. Therefore, with the medical information processing apparatus 120 according to the present embodiment, it is possible to present more appropriate medical procedure as a ToDo list.

If it is not possible to specify whether the medical procedure satisfies the condition to be presented as a ToDo list, from the text data acquired by the second acquisition function 127c, the calculation function 127g may cause a user (participant of the meeting) to input whether the condition is satisfied, after the termination button is pressed.

Moreover, the medical resource information is information on medical resources possessed by the medical facilities. As an example, the medical resource information is information indicating a relation between the amount of medical resources (manpower, supplies, rooms, bed reservations, medical expenses, and the like) at the time point when the importance degree is calculated, and the required amount of medical resources set in advance for each word element that is capable of becoming a candidate for the ToDo list.

In the present embodiment, the calculation function 127g sets fourth weight to be used for calculating the importance degree, according to the amount of medical resources.

Specifically, if the difference between the amount of medical resources at the time point when the importance degree is calculated and the required amount of medical resources is less than a predetermined threshold value, the calculation function 127g sets the value of the fourth weight to be small. On the other hand, if the difference between the amount of medical resources at the time point when the importance degree is calculated and the required amount of medical resources is equal to or greater than a predetermined threshold value, the calculation function 127g sets the value of the fourth weight to be large.

In this manner, by calculating the importance degree while taking into account the medical resource information, for example, the medical procedure that cannot be performed in the first place, due to insufficient medical resources such as manpower is less likely presented as a ToDo list. Therefore, with the medical information processing apparatus 120 according to the present embodiment, it is possible to present more appropriate medical procedure as a ToDo list. Furthermore, if the extracted word element represents the disease name or information on the condition, the weight to be presented as a ToDo list may be set large, by extracting the seriousness of the patient based on the word element. This seriousness may be extracted, by holding a dictionary file in which the word elements and seriousness are associated with each other in advance, and referring to the dictionary file.

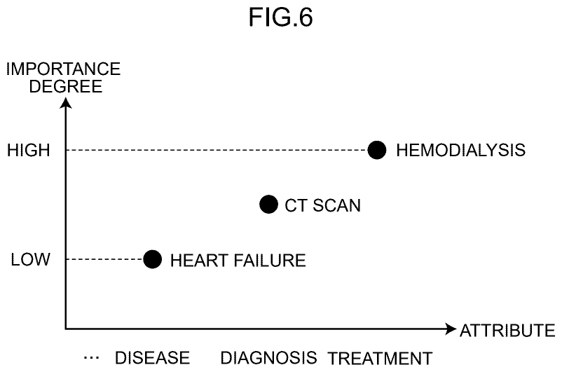

Herein, FIG. 6 is a diagram for explaining an example of calculation results of the importance degree. In FIG. 6, the horizontal axis represents attributes, and the vertical axis represents importance degree. In the example in FIG. 6, the importance degree descends in the order of “hemodialysis” whose word attribute is “treatment”, “CT scan” whose word attribute is “diagnosis”, and “heart failure” whose word attribute is “disease”.

In the present embodiment, the calculation function 127g corrects the importance degree of the calculated word element, while taking into account all of the word attribute, the speaker attribute, the temporal relation, and the medical resource information. However, the calculation function 127g may also correct the importance degree, while taking into account one of the above, or taking into account a combination of two or three of the above.

Moreover, in the present embodiment, the calculation function 127g calculates the importance degree of all of the word attributes. However, the calculation function 127g may calculate the importance degree only for the word attribute representing the medical procedure that may be presented as a ToDo list, such as “treatment” and “diagnosis”.

Returning to FIG. 1, the description will continue. The presentation function 127h presents a candidate for the medical procedure to be performed on the patient, on the basis of the word element according to the calculated importance degree. For example, the presentation function 127h presents the medical procedure according to a predetermined number (for example, three pieces) of word elements as a ToDo list, in a descending order of the importance degree, by displaying the medical procedures on the display 124.

Specifically, the presentation function 127h presents the word attribute representing the medical procedure such as “treatment” and “diagnosis” that may be presented as a ToDo list, as a ToDo list without changing. On the other hand, for the word attribute that cannot be presented as a ToDo list without changing, the presentation function 127h presents the word attribute after converting the attribute into the expression indicating the medical procedure, as a ToDo list.

As an example, in the case of “low blood pressure” whose word attribute is “diagnosis”, by using known natural language processing techniques, the presentation function 127h specifies the predicate of the “low blood pressure” on the basis of the dependency relation, from the text data generated by the generation function 127d. For example, if the specified predicate is “improve”, the presentation function 127h presents the medical procedure of “improve low blood pressure”, as a ToDo list.

Moreover, in the case of “heart failure” whose word attribute is “disease”, similar to the above, the presentation function 127h specifies the predicate of the “heart failure”. For example, if the specified predicate is “evaluate”, the presentation function 127h presents the medical procedure of “evaluate heart failure”, as a ToDo list.

As another example, the presentation function 127h may present the medical procedure that co-occurs as a ToDo list, on the basis of the co-occurrence relation of the word element. For example, if “low blood pressure” and “intubation” are in a co-occurrence relation, the presentation function 127h may present the medical procedure of “intubation” with the word element of “low blood pressure”, as a ToDo list.

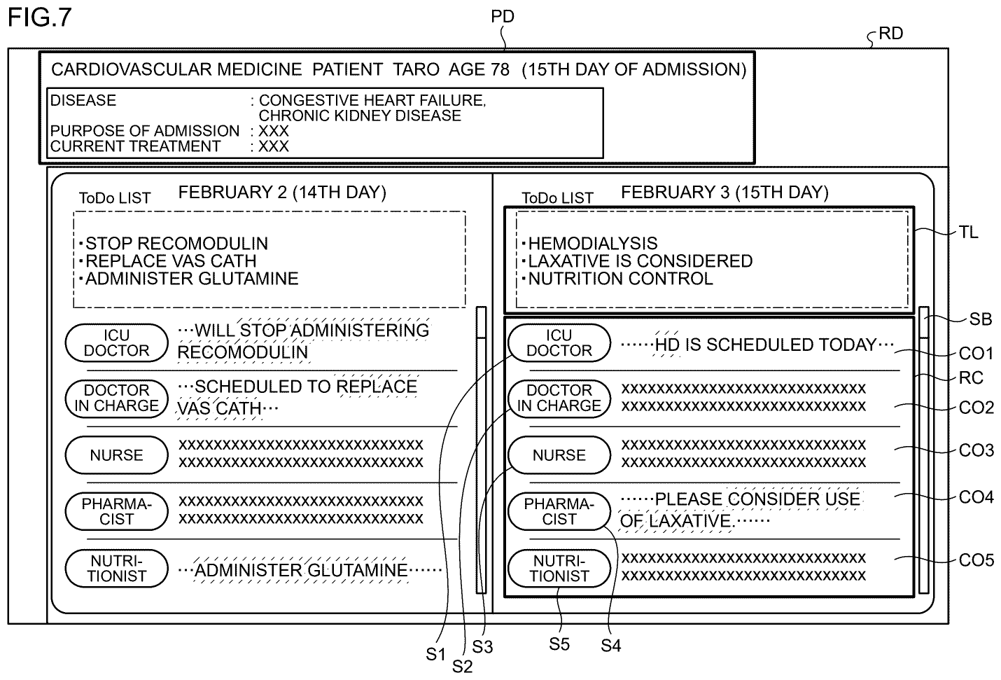

Herein, FIG. 7 is a diagram illustrating an example of a presentation screen RD of a ToDo list. The presentation screen RD in FIG. 7 presents a ToDo list of a target patient by date when the meeting took place. The presentation screen RD in FIG. 7 includes a patient information display field PD, a ToDo list display field TL, and a statement display field RC.

The patient information display field PD is a display field for displaying the medical department, patient name, age, the number of days admitted, disease, purpose of admission, and current treatment, acquired by the first acquisition function 127b as patient information on the target patient.

The medical department is information indicating the medical department of the target patient. The patient name is information indicating the name of the target patient. The age is information indicating the age of the target patient. The number of days admitted is information indicating the number of days the target patient is admitted in the ICU. The disease is information indicating the disease of the target patient. The purpose of admission is information indicating the reason why the target patient was admitted to the ICU. The current treatment is information indicating the treatment currently administered to the target patient.

The ToDo list display field TL is a display field for displaying the ToDo list presented by the presentation function 127h. In the example in FIG. 7, “hemodialysis”, “consideration of laxative”, “nutrition control” that are medical procedures according to the three word elements in a descending order of the importance degree are displayed as a ToDo list. Moreover, if the meeting is currently in progress, the presentation function 127h updates the ToDo list at real time.

Specifically, if the importance degree of the word element calculated by the calculation function 127g at real time exceeds at least one of the importance degrees of the word elements serving as the base of the medical procedures displayed as a ToDo list at the present time, the presentation function 127h updates the ToDo list so that the medical procedures according to the three word elements are displayed in a descending order of the importance degree at the present time.

The statement display field RC is a display field for displaying the statements of the participants during the meeting. In the statement display field RC, statements CO1 to CO5 are displayed in association with information S1 to S5 indicating the speakers. In the example in FIG. 7, the statement CO1 represents the statement of the ICU doctor S1, the statement CO2 represents the statement of the doctor in charge S2 of the medical department, the statement CO3 represents the statement of the nurse S3, the statement CO4 represents the statement of the pharmacist S4, and the statement CO5 represents the statement of the nutritionist S5.

If the statements no longer fit into the statement display field RC, the user can operate a slider bar SB to display the statements given before the statements displayed on the statement display field RC are made. Moreover, when the meeting is currently in progress, the text data generated by the generation function 127d is displayed with information indicating the speaker specified by the calculation function 127g as described above at real time.

During the time from when the reception function 127a receives input to start the meeting to when the reception function 127a receives input to terminate the meeting, the generation function 127d, the extraction function 127e, the specification function 127f, the calculation function 127g, and the presentation function 127h perform the process described above on the voice data acquired by the second acquisition function 127c. After finishing the series of processes, the circuitry 127 stores the ToDo list in the ToDo list display field TL, and the statements in the statement display field RC, in the record of rounds DB 122a as a record of rounds.

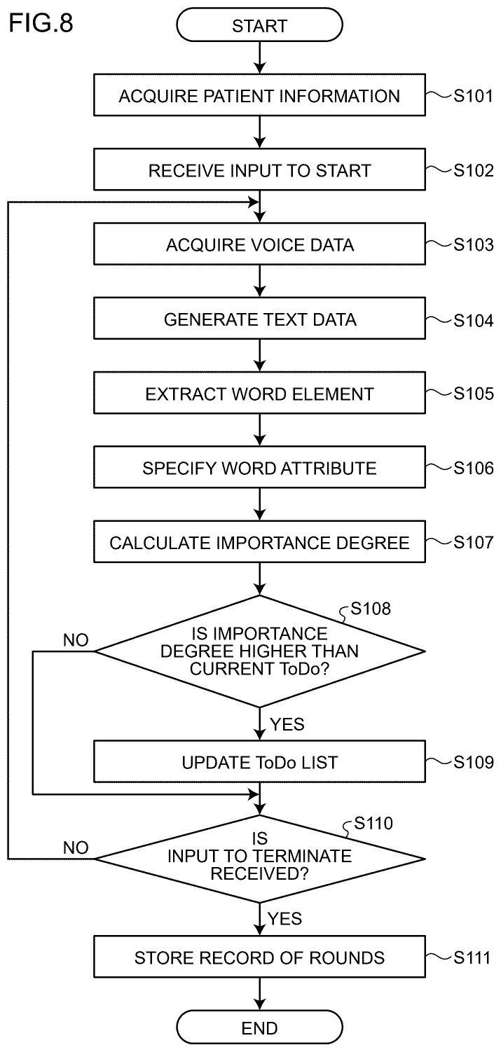

Next, a process executed by the medical information processing apparatus 120 according to the present embodiment will be described. FIG. 8 is a flowchart illustrating an example of a process executed by the medical information processing apparatus 120.

First, the first acquisition function 127b acquires the patient information on the target patient (step S101).

For example, the reception function 127a receives input of the patient ID of the target patient from the user. The first acquisition function 127b refers to the patient information DB 111 of the information integration system 110 via the NW interface 121 and the network 130, and acquires the patient information corresponding to the patient ID received by the reception function 127a. The patient information acquired by the first acquisition function 127b is displayed on the presentation screen of the ToDo list by the circuitry 127.

Next, the reception function 127a receives input to start the meeting (step S102). For example, the reception function 127a receives input to press the meeting start button displayed on the display 124 via the input interface 123.

Next, the second acquisition function 127c acquires voice data (step S103). For example, the second acquisition function 127c acquires voice data recorded by the microphone 125. Next, the generation function 127d generates text data (step S104). For example, by using known voice recognition techniques, the second acquisition function 127c converts the voice data acquired at step S103 into text data.

Next, the extraction function 127e extracts word elements from the text data (step S105). For example, by using known natural language processing techniques, the extraction function 127e segments a sentence included in the text data generated at step S104 into words. Moreover, for the segmented words, the extraction function 127e performs fluctuation correction to integrate synonyms into the same expression.

Next, the specification function 127f specifies the word attribute for the word element (step S106). For example, for each of the word elements extracted at step S104, by using a known medical ontology, the specification function 127f acquires hypernyms in the medical ontology. The specification function 127f specifies each of the acquired hypernyms as a word attribute for each of the word elements.

Next, the calculation function 127g calculates the importance degree of the word element (step S107). For example, the calculation function 127g calculates the importance degree, on the basis of an index such as the appearance frequency of the word element, and the number of times the medical procedure according to the word element was used as a ToDo list. Moreover, the calculation function 127g corrects the importance degree, on the basis of the word attribute of the word element, the speaker attribute according to the word element, the temporal relation, and the medical resource information.

Next, the presentation function 127h determines whether the importance degree calculated at step S106 exceeds the importance degree of the word element serving as the base of the medical procedure currently presented as a ToDo list (step S108). If the importance degree does not exceed the importance degree of the word element serving as the base of the medical procedure currently presented as a ToDo list (No at step S108), the process proceeds to step S110, which will be described below.

On the other hand, if the importance degree exceeds the importance degree of the word element serving as the base of the medical procedure currently presented as a ToDo list (Yes at step S108), the presentation function 127h updates the ToDo list (step S109). For example, the presentation function 127h updates the ToDo list so that the medical procedures according to the three word elements are displayed in a descending order of the importance degree at the present time.

Next, the reception function 127a determines whether input to terminate the meeting is received (step S110). For example, the reception function 127a determines whether input to press the meeting termination button displayed on the display 124 is received, via the input interface 123. If input to terminate the meeting is not received (No at step S110), the process returns to the step S103 described above.

On the other hand, if input to terminate the meeting is received (Yes at step S110), the circuitry 127 stores the record of rounds (step S111), and terminates the present process. For example, the circuitry 127 performs a process of storing the ToDo list displayed on the presentation screen and the content of a statement made by a speaker, in the record of rounds DB 122a of the memory 122, as a record of rounds. When displaying the ToDo list on the presentation screen, information serving as the base for listing up, may be displayed for each item in the ToDo list. For a certain item in the ToDo list, the word element of high importance serving as the base for extracting the item may be extracted, and displayed beside the item. Moreover, the voice data of the conversation from which the word element is extracted and the relevant part of the text data may be displayed side by side. Consequently, it is possible to know from which conversation the item in the ToDo list is suggested.

As described above, the medical information processing apparatus 120 according to the embodiments generates text data from voice data in which the content of statements made by a plurality of speakers on a subject is recorded; extracts a word element from the generated text data; based on the concept indicated by each of a plurality of the extracted word elements, specifies a word attribute for each of the word elements; based on the word attribute, a speaker attribute of the speaker according to the word element, a temporal relation, and medical resource information, calculates the importance degree of each of the word elements; and based on the word element according to the importance degree, presents a candidate for a medical procedure to be performed on a patient.

Consequently, it is possible to calculate the importance degree of a word element in which the word attribute, the speaker attribute of the speaker according to the word element, the temporal relation, and the medical resource information are taken into account. Thus, for example, it is possible to reduce the possibility of presenting the medical procedure that has already been executed as a candidate for the medical procedure to be performed on the patient, or presenting the medical procedure that cannot be executed in the first place due to insufficient manpower or the like as a candidate for the medical procedure to be performed on the patient. Therefore, the medical information processing apparatus 120 according to the present embodiment can present information important for determining the next medical procedure to be performed on the patient, in an easy-to-understand manner. Moreover, by presenting information in an easy-to-understand manner, it is possible to easily record information, and effectively share information among multiple professionals. In other words, with the medical information processing apparatus 120 according to the present embodiment, it is possible to effectively record and share information important for determining the next medical procedure to be performed on a patient.

The embodiment described above may be modified and implemented as appropriate, by changing a part of the configuration or functions of each apparatus. Therefore, in the following, some modifications according to the embodiment described above will be described as other embodiments. In the following, points different from those in the embodiment described above will be mainly described, and detailed description of points common to the contents already described will be omitted. Moreover, the modifications described below may be implemented individually or in combination as appropriate.

### First Modification

In the embodiments described above, by the presentation function 127h, only information indicating the medical procedure is presented as a ToDo list. In the present modification, information indicating “who” and “when”, “the medical procedure is to be performed” is presented as a ToDo list.

In the present modification, in calculating the importance degree, the calculation function 127g analyzes the text data generated by the generation function 127d using known natural language processing techniques, and specifies “who” and “when” the medical procedure according to the word element the importance degree of which is to be calculated, is to be performed (was performed).

Then, the calculation function 127g compares the amount of medical resources at the time point corresponding to the specified “when” and the required amount of medical resources, and sets the fourth weight based on the medical resource information. Consequently, it is possible to calculate the importance degree of the word element, by evaluating the amount of medical resources more accurately.

Moreover, if the calculation function 127g cannot specify at least one of “who” and “when” from the text data, by using a confirmation-required ToDo list that is different from a normal ToDo list, the presentation function 127h leaves the items of “who” and “when” blank, presents a medical procedure with an input field where a user can fill in, and prompts the user to fill in the input field.

In this case, the reception function 127a receives input of “who” and “when” from the user, and according to the input content, the calculation function 127g calculates the importance degree of the word element. When the importance degree is calculated, the presentation function 127h executes a process of deleting the medical procedure according to the word element, from the confirmation-required ToDo list.

When the medical procedure taking the medical resources commonly used is scheduled to be performed on another patient other than the target patient, in a specified time zone or a time zone corresponding to the input “when”, the presentation function 127h may make a notification that prompts the user to confirm the schedule. Consequently, the user can reconsider the schedule of the medical procedure, according to the state of the target patient and the other patient.

According to the present modification, it is possible to present the next medical procedure to be performed on a patient in a more specific form.

### Second Modification

In the embodiments described above, the medical information processing apparatus 120 executes the process described above via the input interface 123, the display 124, and the microphone 125 provided therein. In the present modification, the process described above is executed by a terminal apparatus used by the participant of the meeting, and a medical information processing apparatus communicably connected to the terminal apparatus.

In the present modification, the terminal apparatus has the same configuration as that of the input interface 123, the display 124, and the microphone 125 described as the components of the medical information processing apparatus 120 in the embodiment described above. For example, the terminal apparatus is implemented by a smartphone, a tablet terminal, a personal computer, a smartphone-like small dedicated device, and the like.

Moreover, the medical information processing apparatus executes the process performed via the input interface 123, the display 124, and the microphone 125 in the embodiment described above, via the terminal apparatus. For example, the medical information processing apparatus is implemented by a computer device such as a server and a workstation.

Then, for example, the terminal apparatus and the medical information processing apparatus are communicatively connected via the Internet. In this case, for example, a medical information processing system may be constructed in the form of cloud computing, in which the medical information processing apparatus provides services according to the requests from the terminal apparatus.

Furthermore, the embodiments described above are examples of cases in which an acquisition unit, an extraction unit, a specification unit, a calculation unit, and a presentation unit in the present specification are implemented by the second acquisition function 127c, the extraction function 127e, the specification function 127f, the calculation function 127g, and the presentation function 127h of the circuitry 127, respectively. However, the embodiments are not limited thereto.

For example, the acquisition unit, the extraction unit, the specification unit, the calculation unit, and the presentation unit in the present specification may be implemented by the second acquisition function 127c, the extraction function 127e, the specification function 127f, the calculation function 127g, and the presentation function 127h of the circuitry 127, respectively, otherwise the same functions may be implemented by hardware only, software only, or a combination of software and hardware.

Moreover, in the embodiments described above, the circuitry 127 is implemented by a single processor. However, the embodiments are not limited thereto. For example, the circuitry 127 may be configured by combining a plurality of independent processors, and each processor may execute a computer program to implement each processing function. Furthermore, each processing function of the circuitry 127 may be appropriately distributed or integrated into single or multiple circuitry.

Still furthermore, each processing function of the circuitry 127 may be implemented by a combination of hardware and software such as a circuit. Still furthermore, in this example, the computer program corresponding to each processing function is stored in a single memory 122. However, the embodiments are not limited thereto. For example, a computer program corresponding to each processing function may be stored in a plurality of memories in a dispersed manner, and the circuitry 127 may read and execute each computer program from each of the memories.

Still furthermore, for example, the term “processor” used in the above embodiments refers to a central processing unit (CPU), a graphics processing unit (GPU), or a circuit such as an application specific integrated circuit (ASIC) and a programmable logic device (for example, a simple programmable logic device (SPLD), a complex programmable logic device (CPLD), and a field programmable gate array (FPGA)).

In this example, instead of storing a computer program in the memory, a computer program may also be directly embedded in a circuit of the processor. In this case, the processor implements the functions by reading and executing a computer program embedded in the circuit. Moreover, each processor in the present embodiments is not limited to being configured as a single circuit for each processor, but may also be configured as a single processor by combining multiple independent circuits to implement the functions.

In this example, a computer program to be executed by the processor is provided by being incorporated in a read only memory (ROM), a memory, and the like in advance. The computer program may also be provided by being recorded on a computer readable non-transitory storage medium such as a compact disc-ROM (CD-ROM), a flexible disk (FD), a CD-Recordable (CD-R), and a digital versatile disc (DVD) in a file format that can be installed or executed on these apparatuses.

Moreover, the computer program may also be stored on a computer connected to a network such as the Internet, and provided or distributed by being downloaded via a network. For example, the computer program is configured by a module including the processing functions described above. As an actual hardware, the CPU reads and executes the computer program from the storage medium such as the ROM, and each module is loaded onto the main storage apparatus, and generated on the main storage apparatus.

Moreover, in the embodiments and modifications described above, each component of each apparatus illustrated in the drawings is functionally conceptual, and need not necessarily be physically configured as illustrated in the drawings. That is, a specific mode of dispersion or integration of each device is not limited to those illustrated in the drawings, and all or part thereof may be configured in a functionally or physically dispersed or integrated manner in any units according to various loads and usage conditions.

Furthermore, all or any part of the processing functions performed by the apparatuses can be implemented by a CPU and a computer program analyzed and executed by the CPU, or can be implemented as hardware by wired logic.

Of the processes described in the above embodiments and modifications, all or part of the processes described as being automatically performed may be manually performed, or all or part of the processes described as being manually performed may be automatically performed with a known method. In addition, the processing procedures, control procedures, specific names, and information including various kinds of data and parameters illustrated in the above document and drawings can be optionally changed unless otherwise specified.

The various types of data handled in the present specification are typically digital data.

According to at least one of the embodiments described above, it is possible to effectively record and share information important for determining the next medical procedure to be performed on a subject.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical reporter comprising circuitry configured to:
receive recorded data made by a plurality of speakers on a subject is recorded;
extract a word element, from recorded data in which content of a statement;
output extracted recorded data by using the recorded data which includes extracted word element with specified speaker;
specify, by using the extracted recorded data, and based on a concept indicated by each of a plurality of the extracted word elements, a word attribute for each of the word elements;
calculate, based on at least one of the word attribute, a speaker attribute of a speaker according to the word element, a temporal relation between time related to the content of the statement including the word element and reference time, and medical resource information on a medical resource according to the word element, an importance degree of each of the word elements; and
generate a report data based on the importance degree of each of the word elements, which presenting a candidate for a medical procedure to be performed on the subject.

2. The medical reporter according to claim 1, wherein the circuitry causes a display apparatus to display information indicating candidates for a predetermined number of the medical procedures in a descending order of the importance degree.

3. The medical information processing apparatus according to claim 1, wherein the circuitry calculates the importance degree by referring the word element of the attribute that is capable of becoming a candidate for the medical procedure.

4. The medical reporter according to claim 3, wherein
the circuitry
calculates the importance degree of all of the word elements, and
presents a candidate for the medical procedure derived from the word element, for the word element of the attribute that is incapable of becoming a candidate for the medical procedure.

5. The medical reporter according to claim 4, wherein the circuitry calculates the importance degree of the word element, while taking into account an appearance frequency of the word element, and number of times the candidate for the medical procedure according to the word element is presented.

6. The medical reporter according to claim 4, wherein
the circuitry
specifies, for the word element of the attribute that is incapable of becoming the candidate for the medical procedure, a predicate element with respect to the word element, from a word dependency relation, and
presents a candidate for the medical procedure derived from the word element and the predicate element.

7. The medical reporter according to claim 4, wherein the circuitry presents, for the word element of the attribute that is incapable of becoming a candidate for the medical procedure, a candidate for the medical procedure derived from the word element, based on a co-occurrence relation of the word element.

8. The medical reporter according to claim 1, wherein
the circuitry
sets weight for calculating the importance degree, based on at least one of the word attribute, the speaker attribute, the temporal relation, and the medical resource information, and
calculates the importance degree using the weight.

9. The medical reporter according to claim 8, wherein the circuitry sets the weight so that the weight increases, as number of times the speaker attribute made a statement on the word element according to the presented candidate for the medical procedure increases.

10. The medical reporter according to claim 8, wherein
the circuitry
sets, for the word element the temporal relation of which is before present time, the weight so that the weight becomes small, and
sets, for the word element the temporal relation of which is after the present time, the weight so that the weight increases, as the temporal relation is closer to the present time.

11. The medical reporter according to claim 10, wherein the circuitry sets, for the word element serving as a base of the candidate for the medical procedure to be executed when a specific condition is satisfied, the weight according to an achievement status of the condition at the present time.

12. The medical reporter according to claim 8, wherein
information indicating a required amount of medical resource set for each of the word elements is included in the medical resource information, and
the circuitry
compares an amount of the medical resource at present time and the required amount of medical resource specified from the word element, and
sets, for the word element in which a difference between the amount of medical resource at the present time and the required amount of medical resource specified from the word element is less than a threshold value, the weight so that the weight becomes smaller than that of the word element having the difference equal to or greater than the threshold value.

13. The medical reporter according to claim 8, wherein
the medical resource includes a human resource, and
the circuitry
analyzes the recorded data, in calculating the importance degree,
specifies a person who executes a medical procedure according to the word element and specifies execution time,
compares an amount of the medical resource at a time point corresponding to the execution time and a required amount of the medical resource specified from the word element, based on the specified person who executes the medical procedure and the specified execution time, and
sets, for the word element in which a difference between the amount of medical resource at the time point corresponding to the execution time and the required amount of medical resource specified from the word element is less than a threshold, the weight so that the weight becomes smaller than that of the word element having the difference equal to or greater than the threshold value.

14. The medical reporter according to claim 13, wherein
the circuitry
makes a notification that prompts, when it is not possible to specify at least one of the person who executes the medical procedure and the execution time from the recorded data, a user to input information corresponding to an unspecified item between the person who executes the medical procedure and the execution time,
receives input of information corresponding to the unspecified item, and
specifies at least one of the person who executes the medical procedure and the execution time, based on the received input.

15. The medical reporter according to claim 14, wherein the circuitry makes a notification that prompts the user to confirm a schedule, when the medical procedure taking the medical resource commonly used is scheduled to be performed on another subject other than the subject, in a time zone corresponding to the specified execution time.

16. A medical reporter system comprising circuitry configured to:
receive recorded data made by a plurality of speakers on a subject is recorded;
extract a word element, from recorded data in which content of a statement;
specify, based on a concept indicated by each of a plurality of the extracted word elements, a word attribute for each of the word elements;
output extracted recorded data by using the recorded data which includes extracted word element with specified speaker;
calculate, by using the extracted recorded data, and based on at least one of the word attribute, a speaker attribute of a speaker according to the word element, a temporal relation between time related to the content of the statement including the word element and reference time, and medical resource information on a medical resource according to the word element, an importance degree of each of the word elements; and
generate report data based on the importance degree of each of the word elements, which presenting a candidate for a medical procedure to be performed on the subject.

17. A medical reporting method performed by a medical information processing apparatus, the medical information processing method, comprising:
receiving recorded data made by a plurality of speakers on a subject is recorded;
extracting a word element, from recorded data in which content of a statement;
specifying, based on a concept indicated by each of a plurality of the extracted word elements, a word attribute for each of the word elements;
outputting extracted recorded data by using the recorded data which includes extracted word element with specified speaker;
calculating, by using the extracted recorded data, and based on at least one of the word attribute, a speaker attribute of a speaker according to the word element, a temporal relation between time related to the content of the statement including the word element and reference time, and medical resource information on a medical resource according to the word element, an importance degree of each of the word elements; and
generate a report data, based on the importance degree of each of the word elements, which presenting a candidate for a medical procedure to be performed on the subject.
